(19) United States
(12) Patent Application Publication
(10) Pub. No.: US 2008/0038307 A1
(43) Pub. Date: Feb. 14, 2008

(54) Biocompatible Coating, Method, and Use of Medical Surfaces

(76) Inventor: Erika Hoffmann, Eschweiler (DE)

Correspondence Address: J C PATENTS, INC., 4 VENTURE, SUITE 250, IRVINE, CA, 92618, US

(21) Appl. No.: 10/598,396

(22) PCT Filed: Feb. 27, 2005
(86) PCT No.: PCT/DE05/00327

§ 371 Date: Aug. 25, 2006

Related U.S. Application Data
(60) Provisional application No. 60/551,761, filed on Mar. 11, 2004.

(30) Foreign Application Priority Data
Feb. 28, 2004 (DE) 102004009850.6

(51) Int. Cl. A61F 2/06; A61F 2/04; A61K 31/337; A61K 31/56; A61L 33/06; A61P 29/00; A61P 43/00

(52) U.S. Cl. 424/423; 427/2.25; 514/182; 514/449; 523/121; 623/1.46

(57) ABSTRACT

The invention relates to medical products having a surface that is at least partially covered by a polymer layer. Said polymer layer is preferably formed by auto-polymerization. Substances containing least one multiple bond, especially unsaturated fatty acids comprising an alkyl chain consisting of preferably between 7 and 50 carbon atoms are polymerized. Other substances which do not participate in the polymerization can be added to the substances participating in the polymerization reaction. Said substances are preferably saturated fatty acids and fatty acid derivatives. The invention also relates to methods for producing such medical products, and to the use of the same.

The implantation of stents using balloon dilatation of occluded blood vessels increasingly established in the last years. Although stents decrease the risk of a renewed vessel occlusion they are until now not capable of preventing such restenoses completely.

An exact conceptual description of restenosis cannot be found in the technical literature. The most commonly used morphologic definition of the restenosis is the one which defines the restenosis after a successful PTCA (percutaneous transluminal coronary angioplasty) as a reduction of the vessel diameter to less than 50% of the normal one. Hence, this is an empirically defined value of which the hemodynamic relevance and its relation to clinical pathology lacks of a massive scientific basis. In practical experience the clinical aggravation of a patient is often viewed as a sign for a restenosis of the formerly treated vessel segment.

There are three different reasons for the restenosis caused by the stent:
a.) During the first period after the implantation the stent surface is in direct contact with the blood and an acute thrombosis can occur which again occludes the blood vessel due to the now present foreign surface.
b.) The implantation of the stent generates vessel injuries which also induce inflammation reactions which play a crucial role for the recovery process during the first seven days in addition to the above mentioned thrombosis. The concurrent processes herein are among others connected with the release of growth factors which initiate an increased proliferation of the smooth muscle cells which rapidly leads to a renewed occlusion of the vessel, because of uncontrolled growth.
c.) After a couple of weeks the stent starts to grow into the tissue of the blood vessel. This means that the stent is totally surrounded by smooth muscle cells and has no contact to the blood anymore. This cicatrization can be too distinctive (neointima hyperplasia) and may lead to not only a coverage of the stent surface but to the occlusion of the total interior space of the stent.

It was tried vainly to solve the problem of restenosis by the coating of the stents with heparin (J. Whörle et al., European Heart Journal 2001, 22, 1808-1816). Heparin addresses as anti coagulant only the first mentioned cause and is moreover able to unfold its total effect only in solution. This first problem is meanwhile almost totally avoidable medicamentously by administration of anti-coagulants. The second and third problem is intended now to be solved by locally inhibiting the growth of the smooth muscle cells on the stent. This is carried out by e.g. radioactive stents or stents the surface of which is covered with biocompatible materials as well as by stents which release pharmaceutically active agents.

U.S. Pat. No. 5,891,108 discloses for example a stent formed hollow, which can contain pharmaceutical active agents in its interior, that can be released throughout a various number of outlets in the stent. Whereas, EP-A-1 127 582 describes a stent that shows ditches of 0.1-1 mm depth and 7-15 mm length on its surface which are suitable for the reception of an active agent. These active agent reservoirs release similarly to the outlets in the hollow stent the contained pharmaceutically active agent in a punctually high concentration and over a relatively long period of time which, however, leads to the fact that the smooth muscle cells are not anymore or only very delayed capable of enclosing the stent. As a consequence the stent is much longer exposed to the blood, what leads again to increased vessel occlusions by thromboses (Liistro F., Colombo A., Late acute thrombosis after Paclitaxel eluting stent implantation. Heart 2001, 86, 262-4).

One approach to this problem is represented by the phosphorylcholine coating of biocompatible surfaces (WO 0101957), as here phosphorylcholine, a component of the erythrocyte cell membrane, shall create a non thrombogeneous surface as a component of the coated non biodegradable polymer layer on the stent. Depending on its molecular weight, thereby the active agent is absorbed by the polymer containing phosphorylcholine layer or adsorbed on the surface.

Phosphorylcholine is accounted to the group of the membrane constituting phosphoglycerides, which consist of a glycerin molecule, that carries on its first and second hydroxyl groups esterified especially longer-chain saturated and unsaturated fatty acids such as the palmitic acid (C16), the stearic acid (C18) and the oleic acid (C18:1), while the third hydroxyl group binds phosphoric acid. The phosphoric acid forms with a second alcohol, e.g. choline, also an ester, which is referred to as the polar head part.

Fatty acids are water-insoluble, oily or fatty substances, besides water, enzymes and carbohydrates, which represent important biomolecules, that serve whether in the form of the triacylglycerins as combustible for the winning of chemical energy and can be stored or which secure the formation and the continuance of the cell in the form of membrane constituting compounds such as the already mentioned phosphoglycerids and sphingolipids.

EP 0 790 823 uses these lipids for example for the preparation of liposomes occluding active agents, which provide in a polymeric drug delivery material for retaining the active agent on the medical surface coated therewith.

The production of the triacylglycerins and of the phospholipids is an extremely active metabolism, which takes place in every cell. The both synthesized fatty acids palmitic acid (C16) and stearic acid (C18) are also the precursors for the wide spread mono-unsaturated fatty acids in animal tissue such as the palmitoleic acid (C16:1) and the oleic acid (C18:1).

All further important unsaturated fatty acids have to be incorporated via the food as essential fatty acids. Amongst other things the linoleic acid, an omega-6 fatty acid is here to be mentioned, which is finally converted by the organism into arachidonic acid (C20) that is of essential importance as a precursor for the synthesis of thromboxanes and prostaglandins, which regulate in turn many miscellaneous important cell functions.

EP 0 404 683 B1 describes the utilization of fatty acids on medical surfaces, which are in contact with blood. The fatty acids and especially the linoleic acid are bound covalently to the used hydrophilic polymer for the improvement of its hemocompatibility. Mentioned examples of use are artificial organs, dialyzers, blood filters and catheters. But the production efforts of this coating system is high and the required coupling substances are not innocuous, so that according to our knowledge such a coating is not yet brought to market. Moreover the fatty acids are bound to the polymer via a spacer, whereas the fatty acids are bound to the spacer via an amide bonding.

WO-03039612 also refers to the known antithrombotic and antiproliferative effect of the unsaturated fatty acids on the cardiovascular system and describes for the first time a coating of stents with purchasable oils such as olive oil, sun flower oil, palm-oil and fish oil and especially of cod-liver oil. The fluid oils used are utilized as antithrombotic coating, whereas also emulsions supplemented with active agents are applied. But there is to be concerned, that it is surely very difficult to homogeneously disperse the fluid oil on a stent and that the stent remains uncoated in a considerable extent. Moreover, the stent loses on the way to its destination point further coating substance, whereby the uncoated areas are getting bigger, and the content of active agent, that is actually available at the target, is extremely difficult to determine in the end.

In addition, the shelf life of the coating and therewith also the availability time of the active agent added is strongly limited via the coating itself, as the matrix dissolves itself after some period of time, whereby the restenosis rate of the uncoated stent used plays a decisive role again.

Object of the present invention is to provide hemocompatible surfaces of medical products. Preferably suchlike surfaces are additionally capable of releasing one or more antiproliferative, antiinflammatoric, antiangiogenic and/or antithrombotic active agents in a controlled way. Object of the present invention is especially to provide stents, which guarantee a continuous controlled ingrowth of the stent into the vessel wall by providing a biocompatible surface as matrix and which do not cause reactions on the present alien surface through their degradation anymore, that otherwise could lead to a re-occlusion of the blood vessel in the long term.

This object is solved by the technical teaching of the independent claims of the present invention. Further advantageous embodiments of the invention are evident from the dependent claims, the description, as well as the examples.

Surprisingly, it was found that substances, which contain at least one linear or branched and one substituted or non-substituted alkyl moiety with at least one multiple bond, can polymerize after their application on the surface of a medical product at the air into a resin, in which for example pharmaceutical active agents can be still included and whereby a biocompatible coating of the medical surface is achieved through the polymerization.

These substances that actively participate in the polymerization reaction, which bear at least one linear or branched and one substituted or non-substituted alkyl moiety with at least one multiple bond, are preferably substances with at least one unsaturated fatty acid moiety.

Accounted to the substances, which contain at least one alkyl moiety with at least one multiple bond, i.e. preferably one unsaturated fatty acid moiety, are for example fatty acids, fatty acid esters, fatty acid derivatives, ethers, diethers, tetraethers, lipids, oils, fats, glycerides, tri-glycerides, glycol esters, glycerin esters as well as mixtures of the aforementioned substances.

The unsaturated alkyl moiety has between 7 and 50, preferred between 10 and 35, further preferred between 14 and 26 and especially preferred between 17 and 23 carbon atoms.

Thereby the alkyl moiety can be branched or non-branched as well as carry further substituents, for example hydroxyl groups, alkoxyl groups, amino groups, thiol groups, ether groups, thioether groups, halogens, nitro groups, carbonyl groups, carboxyl groups, amide groups, ester groups and other pharmacologically suitable functional groups.

Additionally, the alkyl moiety features at least one multiple bond, i.e. a double or triple bond, whereas substances with a single double bond are preferred. However, the alkyl moiety can be also poly-unsaturated, contain conjugated or isolated double and/or triple bonds or feature a mixture of double or triple bonds, whereas the unsaturated bond(s) can be contained also in a branch or side chain(s) of the alkyl moiety.

According to invention these substances participating in the polymerization reaction, which contain at least one alkyl moiety or fatty acid moiety with at least one multiple bond, are polymerized with each other via exposure to heat, light and/or aerial oxygen through this at least one multiple bond. In this polymerization a catalyst can be used in a biologically and pharmacologically, respectively, suitable concentration. It is especially advantageous, if the substances containing at least one alkyl moiety with at least one multiple bond are capable of auto-polymerization.

Into this matrix generated during the polymerization another miscellaneous substances can be brought in, which do not actively participate in the polymerization, but are occluded in the generated polymer matrix. These are described more below.

The preferred substances participating in the polymerisation can be represented by the following general formulas: 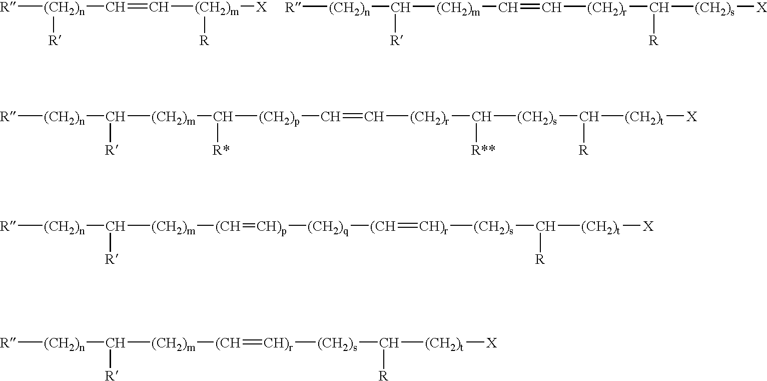
wherein
R, R′, R″, R* and R** represent independently from each other alkyl-, alenyl-, alkinyl-, heteroalkyl-, cycloalkyl-, heterocyclyl moieties with 1 to 20 carbon atoms, aryl-, arylalkyl-, alyklaryl-, heteroaryl moieties with 3 to 20 carbon atoms or represent functional groups and mean preferably the following moieties: —H, —OH, —OCH₃, —C₂H₅, —OC₃H₇, —O-cyclo-C₃H₅, —OCH(CH₃)₂, —OC(CH₃)₃, —OC₄H₉, —OPh, —OCH₂-Ph, —OCPh₃, —SH, —SCH₃, —SC₂H₅, —NO₂, —F, Cl, —Br, —I, —CN, —OCN, —NCO, —SCN, —NCS, —CHO, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —COO-cyclo-C₃H₅, —COOCH(CH₃)₂, —COOC(CH₃)₃, —OOC—CH₃, —OOC—C₂H₅, —OOC—C₃H₇, —OOC-cyclo-C₃H₅, —OOC—CH(CH₃)₂, —OOC(CH₃)₃, —CONH₂, —CONHCH₃, —CONHC₂H₅, —CONHC₃H₇, —CON(CH₃)₂, —CON(C₂H₅)₂, —CON(C₃H₇)₂, —NH₂, —NHCH₃, —NHC₂H₅, —NHC₃H₇, —NH-cyclo-C₃H₅, —NHCH(CH₃)₂, —NHC(CH₃)₃), —N(CH₃)₂, —N(C₂H₅)₂, —N(C₃H₇)₂, —N(cyclo-C₃H₅)₂, —N[CH(CH₃)₂]₂, —N[C(CH₃)₃]₂, —SOCH₃, —SOC₂H₅, SOC₃H₇, —SO₂CH₃, —SO₂C₂H₅, —SO₂C₃H₇, —SO₃H, —SO₃CH₃, —SO₃C₂H₅, —SO₃C₃H₇, —OCF₃, —OC₂F₅, —O—COOCH₃, —O—COOC₂H₅, —O—CO—OC₃H₇, —O—CO—O-cyclo-C₃H₅, —O—CO—OCH(CH₃)₂, —O—COOC(CH₃)₃, —NH—CO—NH₂, —NH—CO—NHCH₃, —NH—CO—NHC₂H₅, —NH—CO—N(CH₃)₂, —NH—CO—N(C₂H₅)₂, —O—CO—NH₂, —O—CO—NHCH₃, —O—CONHC₂H₅, —O—CO—NHC₃H₇, —O—CO—N(CH₃)₂, —O—CO—N(C₂H₅)₂, —O—CO—OCH₃, —O—CO—OC₂H₅, —O—CO—OC₃H₇, —O—CO—O-cyclo-C₃H₅, —O—CO—OCH(CH₃)₂, —O—CO—OC(CH₃)₃, —CH₂F, —CHF₂, —CF₃, —CH₂Cl, —CH₂Br, —CH₂I, —CH₂—CH₂F, —CH₂—CHF₂, —CH₂—CF₃, —CH₂—CH₂Cl, —CH₂—CH₂Br, —CH₂—CH₂I, —CH₃, —C₂H₅, —C₃H₇-cyclo-C₃H₅₇—CH(CH₃)₂, —C(CH₃)₃, C₄H₉, —CH₂—CH(CH₃)₂, —CH(CH₃)—C₂H₅, -Ph, —CH₂-Ph, —CPh₃, —CH═CH₂, —CH₂—CH═CH₂, —C(CH₃)═CH₂, —CH═CH—CH₃—C₂H₄—CH═CH₂, —CH═C(CH₃)₂, —C≡CH, —C≡C—CH₃, —CH₂—C≡CH;
X represents an ester group or amide group and means especially —O-alkyl, —O—CO-alkyl, —O—CO—O-alkyl, —O—CO—NH-alkyl, —O—CO—N-dialkyl, —CO—NH-alkyl, —CO—N-dialkyl, —CO—O-alkyl, —CO—OH, —OH;
m, n, p, q, r, s and t mean independently from each other integers from 0 to 20, preferably from 0 to 10.

The term “alkyl” such as in the case of —CO—O-alkyl means preferably one of the alkyl moieties mentioned for the afore-mentioned moieties R, R′ etc., e.g. —CH₂-Ph. The compounds of the afore-mentioned general formulas also can be present in form of their salts, as racemates or diastereomeric mixtures, as pure enantiomers or diastereomers as well as mixtures or oligomers or copolymers or block-copolymers. Further the afore-mentioned compounds can be used in the mixture with substances not participating in the polymerisation and especially in the mixture with the herein mentioned oils and/or fatty acids. Preferred are such mixtures and individual substances which are suitable for polymerisation, especially for auto-polymerisation.

The substances participating in the polymerization comprise inter alia oils, fats, fatty acids as well as fatty acid esters, which are described in more detail below.

In the case of the lipids are preferably concerned mono- or poly-unsaturated fatty acids and/or mixtures of these unsaturated fatty acids in the form of their tri-glycerides and/or in non glycerin bound, free form.

Preferably the unsaturated fatty acids are chosen from the group, which comprises oleic acid, eicosapentaenoic acid, timnodonic acid, docosahexaenoic acid, arachidonic acid, linoleic acid, α-linolenic acid, γ-linolenic acid as well as mixtures of the aforementioned fatty acids. These mixtures comprise especially mixtures of the pure unsaturated compounds.

As oils are preferably used linseed oil, hempseed oil, corn oil, walnut oil, rape oil, soy bean oil, sun flower oil, poppy-seed oil, safflower oil (Färberdistelöl), wheat germ oil, safflor oil, grape-seed oil, evening primrose oil, borage oil, black cumin oil, algae oil, fish oil, cod-liver oil and/or mixtures of the aforementioned oils. Especially suitable are mixtures of the pure unsaturated compounds.

Fish oil and cod-liver oil mainly contain eicosapentaenoic acid (EPA C20:5) and docosahexaenoic acid (DHA C22:6) besides of little α-linolenic acid (ALA C18:3). In the case of all of the three fatty acids, omega-3 fatty acids are concerned, which are required in the organism as important biochemical constituting substance for numerous cell structures (DHA and EPA), for example as already mentioned, they are fundamental for the build up and continuance of the cell membrane (sphingolipids, ceramides, gangliosides).

Omega-3 fatty acids can be found not only in fish oil, but also in vegetable oils. Further unsaturated fatty acids, such as the omega-6 fatty acids, are present in oils of herbal origin, which here partly constitute a higher proportion than in animal fats. Hence different vegetable oils such as linseed oil, walnut oil, flax oil, evening primrose oil with accordingly high content of essential fatty acids are recommended as especially high-quality and valuable edible oils. Especially linseed oil represents a valuable supplier of omega-3 and omega-6 fatty acids and is known for decades as high-quality edible oil.

As participating substances in the polymerization reaction the omega-3 as well as the omega-6 fatty acids are preferred as well as all of the substances, which bear at least one omega-3 and/or omega-6 fatty acid moiety. Suchlike substances demonstrate also a good capability for auto-polymerization.

The ability of curing, i.e. the ability for auto-polymerization, is based in the composition of the oils, also referred to as toweling oils, and goes back to the high content of essential fatty acids, more precisely to the double bonds of the unsaturated fatty acids. Exposed to air radicals are generated by means of the oxygen on the double bond sites of the fatty acid molecules, which initiate and propagate the radical polymerization, such that the fatty acids cross link among themselves under loss of the double bonds. With the clearing of the double bond in the fat molecule the melting point increases and the cross linking of the fatty acid molecules causes an additional curing. A high molecular resin results, which covers the medical surface homogeneously as flexible polymer film.

The auto-polymerization is also referred to as self-polymerization and can be initiated for example by oxygen, especially by aerial oxygen. This auto-polymerization can also be carried out under exclusion of light. Another possibility exists in the initiation of the auto-polymerization by electromagnetic radiation, especially by light. Still another but less preferred variant is represented by the auto-polymerization initiated by chemical decomposition reactions, especially by decomposition reactions of the substances to be polymerized.

The more multiple bonds are present in the fatty acid moiety, the higher is the degree of cross-linking. Thus, the higher the density of multiple bonds is in an alkyl moiety (fatty acid moiety) as well as in one molecule, the smaller is the amount of substances, which participate actively in the polymerization reaction.

The content of substances participating actively in the polymerization reaction in respect to the total amount of all of the substances deposited on the surface of the medical product is at least 25% by weight, preferred 35% by weight, more preferred 45% by weight and especially preferred 55% by weight.

The following table 1 shows a listing of the fatty acid constituents in different oils, which are preferably used in the present invention.

TABLE 1

| | | | | Eicosa- | Docosa- |
|---|---|---|---|---|---|
| | | Linoleic | Linolenic | pentaenoic | hexaenoic |
| | Oleic acid | acid | acid | acid | acid |
| | (C 18:1) | (C 18:2) | (C 18:3) | (C 20:5) | (C 22:6) |
| Oil species | omega-9 | omega-6 | omega-3 | omega-3 | omega-3 |
| Olive oil | 70 | 10 | 0 | 0 | 0 |
| Corn oil | 30 | 60 | 1 | 0 | 0 |
| Linseed oil | 20 | 20 | 60 | 0 | 0 |
| Cod-liver oil | 25 | 2 | 1 | 12 | 8 |
| Fish oil | 15 | 2 | 1 | 18 | 12 |

The oils and mixtures of the oils, respectively, used in the coating according to invention contain an amount of unsaturated fatty acids of at least 40% by weight, preferred an amount of 50% by weight, more preferred an amount of 60% by weight, further preferred an amount of 70% by weight and especially preferred an amount of 75% by weight of unsaturated fatty acids. Should commercially available oils, fats or waxes be used, which contain a lower amount of compounds with at least one multiple bond than 40% by weight, so unsaturated compounds can be added in the quantity, that the amount of unsaturated compounds increases to over 40% by weight. In the case of an amount of less than 40% by weight the polymerization rate decreases too strong, so that homogeneous coatings cannot be guaranteed any more.

The property to polymerize empowers especially the lipids with high amounts of poly-unsaturated fatty acids as excellent substances for the present invention.

So the linoleic acid (octadecadienoic acid) possesses two double bonds and the linolenic acid (octadecatrienoic acid) possesses three double bonds. Eicosapentaenoic acid (EPA C20:5) has five double bonds and docosahexaenoic acid (DHA C22:6) has six double bonds in one molecule. With the number of double bonds also the readiness to the polymerization increases. These properties of the unsaturated fatty acids and of their mixtures as well as their tendency for auto-polymerization can be used for the biocompatible and flexible coating of medical surfaces especially of stents with e.g. fish oil, cod-liver oil or linseed oil.

Linoleic acid is also referred to as cis-9, cis-12-octadecadienoic acid (chemical nomenclature) or as Δ9,12-octadecadienoic acid or as octadecadienoic acid (18:2) and octadecadienoic acid 18:2 (n-6), respectively, (biochemical and physiological nomenclature, respectively). In the case of octadecadienoic acid 18:2 (n-6) n represents the number of carbon atoms and the number “6” indicates the position of the final double bond. Thus, 18:2 (n-6) is a fatty acid with 18 carbon atoms, two double bonds and with a distance of 6 carbon atoms from the final double bond to the external methyl group.

Preferably used are for the present invention the following unsaturated fatty acids as substances, which participate in the polymerization reaction and substances, respectively, which contain these fatty acids, or substances, which contain the alkyl moiety of these fatty acids, i.e. without the carboxylate group (—COOH).

TABLE 2
Monoolefinic fatty acids

| Systematic name | Trivial name | Short form |
|---|---|---|
| cis-9-tetradecenoic acid | myristoleic acid | 14:1(n-5) |
| cis-9-hexadecenoic acid | palmitoleic acid | 16:1(n-7) |
| cis-6-octadecenoic acid | petroselinic acid | 18:1(n-12) |
| cis-9-octadecenoic acid | oleic acid | 18:1(n-9) |
| cis-11-octadecenoic acid | vaccenic acid | 18:1(n-7) |
| cis-9-eicosenoic acid | gadoleinic acid | 20:1(n-11) |
| cis-11-eicosenoic acid | gondoinic acid | 20:1(n-9) |
| cis-13-docosenoic acid | erucinic acid | 22:1(n-9) |
| cis-15-tetracosenoic acid | nervonic acid | 24:1(n-9) |
| t9-octadecenoic acid | elaidinic acid | |
| t11-octadecenoic acid | t-vaccenic acid | |
| t3-hexadecenoic acid | | trans-16:1 (n-13) |

TABLE 3
Poly-unsaturated fatty acids

| Systematic name | Trivial name | Short form |
|---|---|---|
| 9,12-octadecadienoic acid | linoleic acid | 18:2(n-6) |
| 6,9,12-octadecatrienoic acid | γ-linolenic acid | 18:3(n-6) |
| 8,11,14-eicosatrienoic acid | dihomo-γ- | 20:3(n-6) |
| | linolenic acid | |
| 5,8,11,14-eicosatetraenoic acid | arachidonic acid | 20:4(n-6) |
| 7,10,13,16-docosatetraenoic acid | — | 22:4(n-6) |
| 4,7,10,13,16-docosapentaenoic acid | — | 22:5(n-6) |
| 9,12,15-octadecatrienoic acid | α-linolenic acid | 18:3(n-3) |
| 6,9,12,15-octadecatetraenoic acid | stearidonic acid | 18:4(n-3) |
| 8,11,14,17-eicosatetraenoic acid | — | 20:4(n-3) |
| 5,8,11,14,17-eicosapentaenoic acid | EPA | 20:5(n-3) |
| 7,10,13,16,19-docosapentaenoic acid | DPA | 22:5(n-3) |
| 4,7,10,13,16,19-docosahexaenoic acid | DHA | 22:6(n-3) |
| 5,8,11-eicosatrienoic acid | meadic acid | 20:3(n-9) |
| 9c,11t,13t-eleostearinoic acid | | |
| 8t,10t,12c-calendinoic acid | | |
| 9c,11t,13c-catalpicoic acid | | |
| 4,7,9,11,13,16,19-docosahepta- | stellaheptaenic | |
| decanoic acid | acid | |
| | taxolic acid | all-cis- |
| | | 5,9-18:2 |
| | pinolenic acid | all-cis- |
| | | 5,9,12-18:3 |
| | sciadonic acid | all-cis- |
| | | 5,11,14-20:3 |

TABLE 4
Acetylenic fatty acids

| Systematic name | Trivial name |
|---|---|
| 6-octadecynoic acid | taririnic acid |
| t11-octadecen-9-ynoic acid | santalbinic or ximeninic acid |
| 9-octadecynoic acid | stearolinic acid |
| 6-octadecen-9-ynoic acid | 6,9-octadeceninic acid |
| t10-heptadecen-8-ynoic acid | pyrulinic acid |
| 9-octadecen-12-ynoic acid | crepenynic acid |
| t7,t11-octadecadiene-9-ynoic acid | heisterinic acid |
| t8,t10-octadecadiene-12-ynoic acid | — |
| 5,8,11,14-eicosatetraynoic acid | ETYA |

After accomplishment of the described polymerization of the substances containing one linear or branched and one substituted or non-substituted alkyl moiety with at least one multiple bond, a surface of a medical product is obtained, which is at least partially provided with one polymer layer. In the ideal case a homogeneous continuously thick polymer layer is formed on the total external surface of the medical product and on the total surfaces of the medical product coming into contact with blood or blood products, respectively. This polymer layer on the surface of the medical product consists of the substances participating in the polymerization reaction and includes the substances in the polymer matrix participating not actively in the polymerization reaction. Preferably the occlusion is adapted to allow the substances not participating in the polymerization, especially the active agents, to diffuse out from the polymer matrix.

The biocompatible coating of the polymerized substances provides for the necessary blood compatibility of the medical product, especially of the stent, and represents at the same time a suitable substrate for active agents. An added active agent (or active agent combination), which is homogeneously dispersed over the total surface of the medical product, especially of a stent, effects, that the population of the surface by cells, especially by smooth muscle and endothelic cells, takes place in a controlled way. Thus, rapid population and overgrowth with cells on the stent surface does not take place, which could lead to restenosis, however the population with cells on the stent surface is not completely prevented by a high concentration of a medicament, which involves the danger of a thrombosis.

Thus, it is guaranteed under active support of the matrix, that the active agent or the active agent combination, bound covalently and/or adhesively to the subjacent layer and/or implemented covalently and/or adhesively into the layer, is released continuously and in small doses, so that the population of the stent surface by cells is not inhibited, however an excessive population is prevented. This combination of both effects awards the ability to the surface of a medical product according to invention, especially to the surface of a stent, to grow rapidly into the vessel wall and reduces both the risk of restenosis and the risk of thrombosis. The release of the active agent or of the active agents spans over a time period from 1 to 12 months, preferably 1 to 2 months after implantation.

As active agents are used antiproliferative substances, antiphlogistic as well as antithrombotic, antimigrative and/or antiangiogenic agents. The active agents are used individually or combined in the same or different concentration as substances non-participating in the polymerization reaction. These active agents can be deposited in the form of a first lower layer on the surface of the medical product and the further substances participating in the polymerization with at least one alkyl moiety with at least one multiple bond as well as the other substances non-participating in the polymerization can be deposited on this active agent layer and can then be polymerized, preferably auto-polymerized. Further the possibility exists to admix the active agents to the substances participating in the polymerization reaction, so that the active agents are occluded in the polymer matrix. By such an occlusion of the active agents it is achieved, that these are released continuously from the polymer matrix over the above described periods of time. The time period of the active agent release can be controlled through the polymerization degree. The higher the polymerization degree, the longer the time period, over which the active agent or the active agents are released. Further there is also the possibility to deposit the active agent or the active agent combination after accomplished polymerization reaction on the polymer matrix on the medical product surface or to incorporate the active agent or the active agents into the matrix after swelling of the polymer matrix. Another embodiment includes the covalent coupling of one or more active agents with the polymer matrix and/or with the substances, which did not participate actively in the polymerization reaction. It is also possible to deposit and incorporate, respectively, one or more active agents under and/or in and/or on the polymer matrix, whether before, during or after the polymerization reaction.

Especially preferred are active agents, which feature besides their antiproliferative effect also immunosuppressive characteristics and which are selected from the groups comprising sirolimus (rapamycin), everolimus, pimecrolimus, somatostatin, tacrolimus, roxithromycin, dunaimycin, ascomycin, bafilomycin, erythromycin, midecamycin, josamycin, concanamycin, clarithromycin, troleandomycin, folimycin, cerivastatin, simvastatin, lovastatin, fluvastatin, rosuvastatin, atorvastatin, pravastatin, pitavastatin, vinblastine, vincristine, vindesine, vinorelbine, etoposide, teniposide, nimustine, carmustine, lomustine, cyclophosphamide, 4-hydroxycyclophosphamide, estramustine, melphalan, ifosfamide, trofosfamide, chlorambucil, bendamustine, dacarbazine, busulfan, procarbazine, treosulfan, temozolomide, thiotepa, daunorubicin, doxorubicin, aclarubicin, epirubicin, mitoxantrone, idarubicin, bleomycin, mitomycin, dactinomycin, methotrexate, fludarabine, fludarabine-5′-dihydrogenphosphate, cladribine, mercaptopurine, thioguanine, cytarabine, fluorouracil, gemcitabine, capecitabine, docetaxel, carboplatin, cisplatin, oxaliplatin, amsacrine, irinotecan, topotecan, hydroxycarbamide, miltefosine, pentostatin, aldesleukin, tretinoin, asparaginase, pegaspargase, anastrozole, exemestane, letrozole, formestane, aminoglutethimide, adriamycin, azithromycin, spiramycin, cepharantin, smc proliferation inhibitor-2w, epothilone A and B, mitoxantrone, azathioprine, mycophenolatmofetil, c-myc-antisense, b-myc-antisense, betulinic acid, camptothecin, PI-88 (sulfated oligosaccharide), melanocyte stimulating hormone (α-MSH), activated protein C, IL-1β inhibitor, thymosine α-1, fumaric acid and its esters, calcipotriol, tacalcitol, lapachol, β-lapachone, podophyllotoxin, betulin, podophyllic acid 2-ethylhydrazide, molgramostim (rhuGM-CSF), peginterferon α-2b, lenograstim (r-HuG-CSF), filgrastim, macrogol, dacarbazine, basiliximab, daclizumab, selectin (cytokine antagonist), CETP inhibitor, cadherines, cytokinin inhibitors, COX-2 inhibitor, NFkB, angiopeptin, ciprofloxacin, camptothecin, fluoroblastin, monoclonal antibodies, which inhibit the muscle cell proliferation, bFGF antagonists, probucol, prostaglandins, 1,11-dimethoxycanthin-6-one, 1-hydroxy-11-methoxycanthin-6-one, scopoletin, colchicine, NO donors such as pentaerythritol tetranitrate and syndnoeimines, S-nitrosoderivatives, tamoxifen, staurosporine, β-estradiol, α-estradiol, estriol, estrone, ethinylestradiol, fosfestrol, medroxyprogesterone, estradiol cypionates, estradiol benzoates, tranilast, kamebakaurin and other terpenoids, which are applied in the therapy of cancer, verapamil, tyrosine kinase inhibitors (tyrphostines), cyclosporine A, paclitaxel and derivatives thereof such as 6-α-hydroxy-paclitaxel, baccatin, taxotere and others, synthetically produced as well as from native sources obtained macrocyclic oligomers of carbon suboxide (MCS) and derivatives thereof, mofebutazone, acemetacin, diclofenac, lonazolac, dapsone, o-carbamoylphenoxyacetic acid, lidocaine, ketoprofen, mefenamic acid, piroxicam, meloxicam, chloroquine phosphate, penicillamine, tumstatin, avastin, D-24851, SC-58125, hydroxychloroquine, auranofin, sodium aurothiomalate, oxaceprol, celecoxib, β-sitosterin, ademetionine, myrtecaine, polidocanol, nonivamide, levomenthol, benzocaine, aescin, ellipticine, D-24851 (Calbiochem), colcemid, cytochalasin A-E, indanocine, nocodazole, S 100 protein, bacitracin, vitronectin receptor antagonists, azelastine, guanidyl cyclase stimulator, tissue inhibitor of metal proteinase-1 and -2, free nucleic acids, nucleic acids incorporated into virus transmitters, DNA and RNA fragments, plasminogen activator inhibitor-1, plasminogen activator inhibitor-2, antisense oligonucleotides, VEGF inhibitors, IGF-1; active agents from the group of the antibiotics such as cefadroxil, cefazolin, cefaclor, cefotaxim, tobramycin, gentamycin, penicillins such as dicloxacillin, oxacillin, sulfonamides, metronidazol, antithrombotics such as argatroban, aspirin, abciximab, synthetic antithrombin, bivalirudin, coumadin, enoxaparin, desulphated and N-reacetylated heparin, tissue plasminogen activator, GpIIb/IIIa platelet membrane receptor, factor Xₐ inhibitor antibodies, heparin, hirudin, r-hirudin, PPACK, protamin, sodium salt of 2-methylthiazolidine-2,4-dicarboxylic acid, prourokinase, streptokinase, warfarin, urokinase, vasodilators such as dipyramidole, trapidil, nitroprussides, PDGF antagonists such as triazolopyrimidine and seramin, ACE inhibitors such as captopril, cilazapril, lisinopril, enalapril, losartan, thio-protease inhibitors, prostacyclin, vapiprost, α, β and γinterferon, histamine antagonists, serotonin blockers, apoptosis inhibitors, apoptosis regulators such as p65, NF-kB or Bcl-xL antisense oligonucleotides, halofuginone, nifedipine, tocopherol, vitamin B1, B2, B6 and B12, folic acid, tranilast, molsidomine, tea polyphenols, epicatechin gallate, epigallocatechin gallate, Boswellinic acids and derivatives thereof, leflunomide, anakinra, etanercept, sulfasalazine, etoposide, dicloxacillin, tetracycline, triamcinolone, mutamycin, procainamid, D24851, SC-58125, retinoic acid, quinidine, disopyramide, flecainide, propafenone, sotalol, amidorone, natural and synthetically produced steroids such as bryophyllin A, inotodiol, maquiroside A, ghalakinoside, mansonine, strebloside, hydrocortisone, betamethasone, dexamethasone, non-steroidal substances (NSAIDS) such as fenoprofen, ibuprofen, indomethacin, naproxen, phenylbutazone and other antiviral agents such as acyclovir, ganciclovir and zidovudine, antimycotics such as clotrimazole, flucytosine, griseofulvin, ketoconazole, miconazole, nystatin, terbinafine, antiprozoal agents such as chloroquine, mefloquine, quinine, moreover natural terpenoids such as hippocaesculin, barringtogenol-C21-angelate, 14-dehydroagrostistachin, agroskerin, agrostistachin, 17-hydroxyagrostistachin, ovatodiolids, 4,7-oxycycloanisomelic acid, baccharinoids B1, B2, B3 and B7, tubeimoside, bruceanol A, B and C, bruceantinoside C, yadanziosides N and P, isodeoxyelephantopin, tomenphantopin A and B, coronarin A, B, C and D, ursolic acid, hyptatic acid A, zeorin, iso-iridogermanal, maytenfoliol, effusantin A, excisanin A and B, longikaurin B, sculponeatin C, kamebaunin, leukamenin A and B, 13,18-dehydro-6-α-senecioyloxychaparrin, taxamairin A and B, regenilol, triptolide, moreover cymarin, apocymarin, aristolochic acid, anopterin, hydroxyanopterin, anemonin, protoanemonin, berberine, cheliburin chloride, cictoxin, sinococuline, bombrestatin A and B, cudraisoflavone A, curcumin, dihydronitidine, nitidine chloride, 12-β-hydroxypregnadiene-3,20-dione, bilobol, ginkgol, ginkgolic acid, helenalin, indicine, indicine-N-oxide, lasiocarpine, inotodiol, glycoside 1a, podophyllotoxin, justicidin A and B, larreatin, malloterin, mallotochromanol, isobutyrylmallotochromanol, maquiroside A, marchantin A, maytansine, lycoridicin, margetine, pancratistatin, liriodenine, oxoushinsunine, aristolactam-AII, bisparthenolidine, periplocoside A, ghalakinoside, ursolic acid, deoxypsorospermin, psychorubin, ricin A, sanguinarine, manwu wheat acid, methylsorbifolin, sphatheliachromen, stizophyllin, mansonine, strebloside, akagerine, dihydrousambarensine, hydroxyusambarine, strychnopentamine, strychnophylline, usambarine, usambarensine, berberine, liriodenine, oxoushinsunine, daphnoretin, lariciresinol, methoxylariciresinol, syringaresinol, umbelliferon, afromoson, acetylvismione B, desacetylvismione A, vismione A and B and sulfur containing amino acids such as cystine as well as salts and/or mixtures of the aforementioned active agents.

Furthermore preferred is a combination of several antiproliferatively acting substances or of antiproliferative active agents with immunosuppressive active agents. Preferred for the present invention are tacrolimus, pimecrolimus, PI-88, paclitaxel and its derivatives, trapidil, α- and β-estradiol, sodium salt of 2-methylthiazolidine-2,4-dicarboxylic acid, macrocyclic carbon suboxide (MCS) and its derivatives, sirolimus, fumaric acid and its esters, activated protein C, interleukin-1β inhibitors and melanocyte-stimulating hormone (α-MSH), cystine, ellipticine, bohemine, indanocine, colcemid and derivatives thereof, methionine as well as salts and/or mixtures of the aforementioned substances.

The active agent is preferably contained in a pharmaceutical active concentration from 0.0001 to 10 mg per cm² medical product surface, especially a stent surface. Further active agents can be contained in similar concentration in the same or in further layers. Preferably the concentration of an active agent on the surface of the medical product is 0.001 to 5 mg per cm² surface, more preferred 0.005 to 3 mg per cm² surface and especially preferred 0.01 to 2 mg per cm² surface of the medical product.

The medical products with a surface coated according to invention can be produced in accordance with the following methods:
a) providing a surface of a medical product, and
b) application of the substances for the polymer layer, and
c) polymerization of the at least one alkyl moiety with substances containing at least one multiple bond by means of exposure to heat, light and/or aerial oxygen and/or by means of one a catalyst contained in a biocompatible concentration.

Thereby the substances for the polymer layer are mixed initially and then applied on the surface of the medical product. To the substances for the polymer layer are accounted the substances participating in the polymerization reaction, i.e. the substances participating actively in the polymerization reaction, which contain at least one alkyl moiety with at least one multiple bond, whereas these substances are linked with each other covalently via the polymerization of this said at least one multiple bond. Moreover, the substances for the polymer layer can further contain substances participating not actively in the polymerization reaction. These substances participating not in the polymerization comprise for example the above described active agents, compounds, which feature one alkyl moiety comparable in the number of carbon atoms and the substituents with the alkyl moiety of the substances participating actively in the polymerization, however with the difference, that the alkyl moiety of the substances participating not in the polymerization features no multiple bonds. In the case of these alkyl moieties preferably saturated fatty acid moieties are concerned. Further not accounted to the substances participating not in the polymerization reaction are saturated fatty acids, saturated fatty acid esters, saturated fatty acid derivatives, saturated ethers, saturated lipids, lipoids, saturated fats and oils, saturated glycerides, saturated triglycerides, saturated glycol esters, saturated glycerin esters, waxes, biostable or biodegradable polymers or mixtures of the aforementioned substances.

As waxes are suitable for example beeswax, carnauba wax, candelilla wax as well as mixtures of these waxes.

Preferably also saturated fatty acids are used, which preferably feature a chain length of at least 12 carbon atoms.

TABLE 5
Saturated fatty acids

| Systematic name | Trivial name | Short form |
|---|---|---|
| dodecanoic acid | laurinic acid | 12:0 |
| tetradecanoic acid | myristinic acid | 14:0 |
| hexadecanoic acid | palmitinic acid | 16:0 |
| heptadecanoic acid | margarinic acid | 17:0 |
| octadecanoic acid | stearinic acid | 18:0 |
| eicosanoic acid | arachinic acid | 20:0 |
| docosanoic acid | behenic acid | 22:0 |
| tetracosanoic acid | lignocerinic acid | 24:0 |

Further preferred are also mixtures of saturated fatty acids and/or natural lipoids such as palm kernel fat and coconut fat.

Especially suitable are the following biostable polymers: polyacrylic acid and polyacrylates such as polymethylmethacrylate, polybutylmethacrylate, polyacrylamide, polyacrylonitriles, polyamides, polyetheramides, polyethylenamine, polyimides, polycarbonates, polycarbourethanes, polyvinyl ketones, polyvinylhalogenides, polyvinylidenhalogenides, polyvinylethers, polyvinylaromates, polyvinylesters, polyvinylpyrollidones, polyoxymethylenes, polyethylene, polypropylene, polytetrafluoroethylene, polyurethanes, polyolefine elastomeres, polyisobutylenes, EPDM gums, fluorosilicones, carboxymethylchitosanes, polyethyleneterephthalate, polyvalerates, carboxymethylcellulose, cellulose, rayon, rayontriacetates, cellulosenitrates, celluloseacetates, hydroxyethylcellulose, cellulosebutyrates, celluloseacetatebutyrates, ethylvinylacetate copolymers, polysulphones, epoxy resins, ABS resins, EPDM gums, silicones such as polysiloxanes, polyvinylhalogenes and copolymers, celluloseethers, cellulosetriacetates, chitosanes and copolymers and/or mixtures of these substances.

As biodegradable polymers are suitable for example polyvalerolactones, poly-ε-decalactones, polylactides, polyglycolides, copolymers of the polylactides and polyglycolides, poly-ε-caprolactone, polyhydroxybutanoic acid, polyhydroxybutyrates, polyhydroxyvalerates, polyhydroxybutyrate-co-valerates, poly(1,4-dioxane-2,3-diones), poly(1,3-dioxane-2-one), poly-para-dioxanones, polyanhydrides such as polymaleic anhydrides, polyhydroxymethacrylates, fibrin, polycyanoacrylates, polycaprolactonedimethylacrylates, poly-b-maleic acid, polycaprolactonebutyl-acrylates, multiblock polymers such as for example from oligocaprolactonedioles and oligodioxanonedioles, polyetherester multiblock polymers such as for example PEG and poly(butyleneterephtalate), polypivotolactones, polyglycolic acid trimethyl-carbonates, polycaprolactone-glycolides, poly(g-ethylglutamate), poly(DTH-iminocarbonate), poly(DTE-co-DT-carbonate), poly(bisphenol-A-iminocarbonate), polyorthoesters, polyglycolic acid trimethyl-carbonates, polytrimethylcarbonates, polyiminocarbonates, poly(N-vinyl)-pyrrolidone, polyvinylalcoholes, polyesteramides, glycolated polyesters, polyphosphoesters, polyphosphazenes, poly[p-carboxyphenoxy)propane], polyhydroxypentanoic acid, polyanhydrides, polyethyleneoxide-propyleneoxide, soft polyurethanes, polyurethanes with amino acid moieties in the backbone, polyetheresters such as polyethyleneoxide, polyalkeneoxalates, polyorthoesters as well as their copolymers, carrageenans, fibrinogen, starch, collagen, protein based polymers, polyamino acids, synthetic polyamino acids, zein, modified zein, polyhydroxyalkanoates, pectic acid, actinic acid, modified and non modified fibrin and casein, carboxymethylsulphate, albumin, moreover hyaluronic acid, heparansulphate, heparin, chondroitinesulphate, dextran, b-cyclodextrines and copolymers with PEG and polypropyleneglycol, gummi arabicum, guar, gelatin, collagen, collagen-N-Hydroxysuccinimide, modifications and copolymers and/or mixtures of the aforementioned substances.

These substances do not participate actively in the polymerization reaction, i.e. they are not bound covalently in the or to the polymer matrix, but they are bound in the polymer matrix in that way, that they could be dissolved out from the matrix without cleavage of a covalent bond. This applies especially for the above-mentioned active agents, which are deposited in the polymer matrix and diffuse out in a controlled way.

After the deposition of the substances for the polymer layer, so a mixture of the substances participating and non participating in the polymerization reaction, the polymer matrix is generated by the polymerization of the substances, which contain at least one alkyl moiety with at least one multiple bond, by means of exposure to heat, light and/or aerial oxygen via this multiple bond. In this polymerization a catalyst can be used in a biocompatible, i.e. pharmacologically suitable concentration. As catalysts come into consideration for example organic radicals or organic compounds, which dissociate into radicals, such as peroxides or diazo compounds. Further also inorganic catalysts such as potassium permanganate, iodine or bromine can be used.

In another method according to invention one layer of an antiproliferative, antiinflammatory and/or antithrombotic active agent or active agent combination of the above-mentioned active agents is applied initially to the deposition of the substances for the polymer layer. On this layer the substances are deposited then for the polymer layer, which can also contain one or more of the above-mentioned active agents, and are then polymerized.

Preferably used for the polymerization reaction are suchlike substances, which auto-polymerize.

After the formation of the polymer layer another active agent layer can be deposited or incorporated on or in this layer. The deposition can be carried out adhesively or also covalently. It is not necessary to use an active agent or an active agent combination, which is already contained in a lower layer or in the polymer layer. A subsequent incorporation of one or more active agents into the polymer layer can be effected by means of swelling of the polymer layer and by diffusion of the active agent(s).

Directly on the polymer layer or preferably on this external active agent layer a further second, third or forth layer of a biostable and/or biodegradable polymer can be deposited. This external layer of one of the above-mentioned biostable or biodegradable polymers serves as protective layer, which allows for a controlled release of the active agents from the subjacent layers.

Instead of this external polymer layer also another layer of substances for the subjacent layer can be deposited according to the process steps b) and c).

The substances for the polymer layer as well as for the optional further polymer layer according to process step b) and c) are deposited by the dipping and/or spraying method. Thereby the active agent or the active agent mixture is admixed to the not completely polymerized spray or dipping solution consisting of the substances for the polymer layer.

The above-mentioned active agents can be bound adhesively and/or covalently to, in, on and/or under a layer.

Thus, the present invention relates also to medical products, the surfaces of which have been coated according to one of the methods according to invention. These medical products are preferably suitable for the direct contact with blood or blood products. Especially concerned with these medical products are stents. Preferably these stents feature not only a hemocompatible surface according to invention, but contain at least one of the aforementioned antiproliferative, antiinflammatory and/or antithrombotic active agents in a pharmaceutically active concentration of 0.0001 to 10 mg per cm² stent surface, preferred 0.001 to 5 mg per cm² surface, more preferred 0.005 to 3 mg per cm² surface and especially preferred 0.01 to 2 mg per cm² stent surface.

The hemocompatible layer covering directly the stent preferably consists of a polymer network of poly-unsaturated fatty acids. These stents are produced by providing conventional normally uncoated stents and depositing preferably adhesively a biocompatible layer, which polymerizes at the air and, if necessary, by adding a catalyst in a concentration non toxic to humans on the stent into a flexible, thin film covering the whole stent homogeneously. If an active agent or an active agent combination is added, this can be effected by admixing into the fatty acid solution or subsequently by diffusing via swelling processes into the already polymerized matrix or by depositing initially to the coating with the fatty acids in a separate work step.

The conventional stents, which can be coated according to the inventive methods, consist of stainless steel, nitinol or other metals and alloys or of synthetic polymers.

Another preferred embodiment of the stents according to invention features a coating, which consists of at least two layers. Also poly-layer systems are used. In the case of suchlike poly-layer systems a layer is referred to as first layer, which is deposited directly on the stent. A layer is referred to as second layer, which is deposited on the first layer, etc.

According to the dual-layer embodiment the first layer consists of a polymerized fatty acid containing layer, which is substantially completely covered by a layer, which contains at least one antiproliferative, antiphlogistic and/or antithrombotic active agent, bound covalently and/or adhesively. Likewise used are also active agent combinations, which mutually support and complement one another in their effect. As polymerizable oils are used herbal and animal fats with high amounts of unsaturated fatty acids. Thereto accounted are linseed oil, hempseed oil, corn oil, rape oil, soy bean oil, sun flower oil, wheat germ oil, safflower oil, grapeseed oil, evening primrose oil, black cumin oil, algae oil, fish oil, cod-liver oil and/or mixtures of the aforementioned substances but also specifically the polymerizable fats underlying these mixtures linolenic acid (ALA), linoic acid, eicosahexaenoic acid (EPA), docosahexaenoic acid (DHA) as pure substances or in any mixture ratio. The layer(s) containing the active agent is (are) deposited slowly by the constituents of the blood, such that the active agent is released according to the velocity of the degradation of the layer or dissolves itself from the matrix according to its elution behavior. By this biological degradation and by the respective active agent release an ongrowth of cells is strongly reduced only for a certain period of time and a targeted controlled ongrowth is enabled, where the external layer has already been degradated widely. The biological degradation of the polymer layer spans advantageously over 1 to 36 months, preferred over 1 to 6 months. In this period of time the important processes of healing take place.

Suchlike stents can be produced by a method of biocompatible coating of stents with the following underlying principle:
a) providing an uncoated stent,
b) substantially completely covering of the surface in the dipping or spraying method with the non polymerized oil or
b′) substantially completely covering and/or incompletely covering in the dipping or spraying method with the non polymerized oil, which contains at least one active agent,
c) polymerization of the deposited layer at the air and at room temperature or at elevated temperature.

Another embodiment of a biocompatible stent is given, if the oil is deposited on the surface and allowed after accomplished polymerization and curing to diffuse with an active agent or an active agent combination by swelling into the coating. In addition a second pure active agent layer can be deposited on the first active agent free or active agent containing lipid layer.

For a successful homogeneous coating the oil is dissolved in an easy to evaporate organic solvent. Catalysts as wells as synthetic polymers, which shall prevent the oil from dripping off the surface before it polymerizes, can be easily added in this way.

The stents according to invention solve both the problem of acute thrombosis (see FIG. 4) and the problem of neointima hyperplasia after a stent implantation. In addition the inventive stents are especially well suited, because of their coating, whether as mono-layer or as poly-layer system, for the continuous release of one or more antiproliferative, immuno-suppressive and/or antithrombotic active agents. Due to this capability of the targeted continuous active agent release in a required amount the inventively coated stents prevent the danger of restenosis.

### EXAMPLES

### Example 1

### Polymerization of 100% Linseed Oil at 80° C.

Linseed oil is deposited as a thin film on a slide and subsequently stored at 80° C. in the drying oven. After two days the polymerization is accomplished. A homogeneous light yellow dry polymer layer is obtained, which adheres well on the surface.

### Example 2

### Polymerization of 100% Linseed Oil at Room Temperature

Linseed oil is deposited as a thin film on a slide and is stored under air and under exposure to UV radiation (Light). After 14 days the polymerization is accomplished and the oil is cured.

### Example 3

### Polymerization of Mixtures of Linseed Oil and Olive Oil (4:1)

A mixture of 80% linseed oil and 20% olive oil is prepared and deposited as a thin film on a slide and stored at 80° C. in the drying oven. Although the oil became solid after 2 days, it still features a sticky surface. In the case of higher amounts of olive oil the liquid consistency remains.

### Example 4

### Biocompatible Coating of Stents with Linseed Oil Under Adding of a Catalyst and a Synthetic Polymer, Especially Polyvinylpyrrolidone

Non expanded stents of medical stainless steel LVM 316 are removed from fat in the ultrasonic bath for 15 minutes with acetone and ethanol and dried at 100° C. in the drying oven. Subsequently the stents are washed with demineralized water over night. About 10 mg of KMnO₄ are dissolved in 500 μl of water and as much as possible PVP is added. The mixture is spread laminarly on a polypropylene substrate and allowed to dry at room temperature over night. From this brittle mixture 2.5 mg are dissolved in 1 ml of chloroform and the resulting solution is sprayed after adding of 10.5 μl of linseed oil with an airbrush spraying pistol (EVOLUTION from Harder & Steenbeck) from a distance of 6 cm on a rotating 18 mm LVM stainless steel stent. Afterwards the coated stent was stored for 24 h at 80° C.

### Example 5

### Adding of Active Agent to a Coated Stent in the Dipping Method

The coated stent of example 4 was dipped into a solution of 600 μg of paclitaxel in 1 ml of ethanol and allowed to swell. After accomplishing the swelling process the stent was extracted and dried.

### Example 6

### Biocompatible Coating of Stents with Linseed Oil and Paclitaxel

Non expanded stents of medical stainless steel LVM 316 are removed from fat in the ultrasonic bath for 15 minutes with acetone and ethanol and dried at 100° C. in the drying oven. Subsequently the stents were washed with demineralized water over night. Linseed oil and paclitaxel (80:20) are dissolved in the mixture ratio of 1:1 in chloroform and then sprayed on the continuously rotating stent. After evaporation of the chloroform in the soft air stream the stent is stored in the drying oven at 80° C.

### Example 7

### Biocompatible Coating of Stents with a 0.25% Ethanol Linseed Oil Spraying Solution

Non expanded stents of medical stainless steel LVM 316 are removed from fat in the ultrasonic bath for 15 minutes with acetone and ethanol and dried at 100° C. in the drying oven. Subsequently the stents were washed with demineralized water over night. A 0.25% by weight spraying solution of linseed oil and ethanol is prepared and continuously sprayed with a spraying pistol on the stent rotating around its axis. The coated stent is dried over night in the drying oven at 70° C. The average coating mass is 0.15 mg±0.02 mg.

### Example 8

### Biocompatible Coating of Stents with an Ethanol Spraying Solution of Linseed Oil and the Synthetic Polymer Polyvinylpyrrolidone (PVP)

After cleaning of the stents as already described in the examples before an ethanol spraying solution is prepared which contains 0.25% linseed oil and 0.1% PVP and continuously sprayed with a spraying pistol on the stent rotating around its axis. Then it is dried over night at 70° C. The average coating mass is 0.2 mg±0.02 mg.

### Example 9

### Biocompatible Coating of Stents with Linseed Oil and the Synthetic Polymer Polyvinylpyrrolidone (PVP) in the Two Layer System with Addition of a Restenosis Inhibiting Active Agent

After cleaning of the stents a first layer of 0.25% by weight of paclitaxel dissolved in chloroform is sprayed on the stent. After drying of this layer at room temperature the second layer of a chloroform solution with 0.25% linseed oil and 0.1% PVP is sprayed on. After drying over night at 70° C. the coating mass is determined to be 0.3 mg±0.02 mg.

### Example 10

### Biocompatible Coating of Stents with Linseed Oil and the Synthetic Polymer Polyvinylpyrrolidone (PVP) in the Two Layer System with Addition of a Restenosis Inhibiting Active Agent

After cleaning of the stents a first layer of 0.25% by weight of linseed oil as well as estradiol and 0.1% PVP dissolved in ethanol is sprayed on the dry stent. After drying of this layer at 70° C. over night the second layer of a chloroform solution with 0.25% linseed oil and 0.1% PVP is sprayed on. After drying over night at 70° C. the coating mass is determined to be 0.37 mg±0.05 mg.

### Example 11

### Biocompatible Coating of Stents with Linseed Oil and α-Linolenic Acid

After cleaning of the stents with acetone and ethanol as previously described a mixture solved in ethanol with 0.20% linseed oil and 0.5% α-linolenic acid is prepared and continuously sprayed on the stent. Then it is dried over night at 70° C. The average coating mass is 0.2 mg±0.02 mg.

### DESCRIPTION OF THE FIGURES

FIGS. 1 and 2

show light microscopic images of stents coated with linseed oil—PVP (0.1%). The coating is also continuous with changing of the design and does not show any agglomerations which easily can arise in the arches, for example.

FIG. 3

shows the elution measurement of β-estradiol from the linseed oil—PVP (0.1%) matrix.

FIG. 4

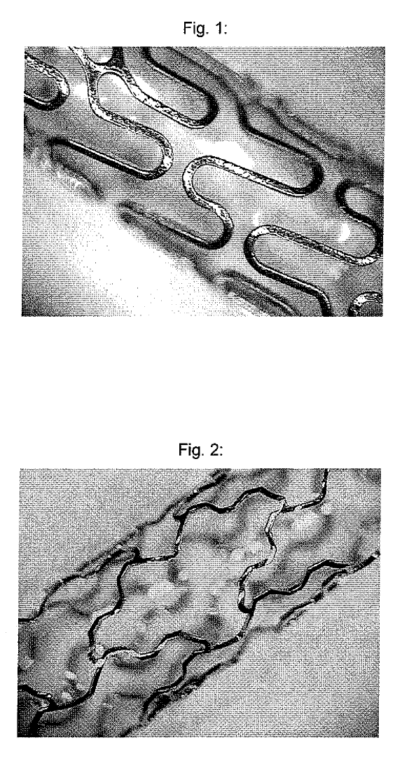

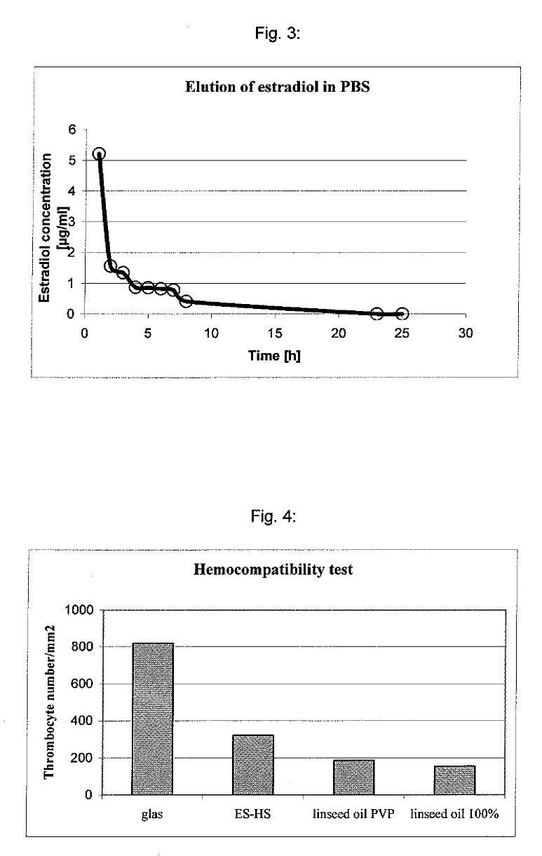

shows the determination of the thrombocyte adhesion on glass, endothelic cell heparan sulphate (ESHS), linseed —PVP (0.1%) and linseed oil (100%) in vitro. The measurement was carried out in the dynamic system of the Baumgartner-chamber (modified according to Sakarassien) with human whole blood.

The linseed matrix with and with out PVP addition is compared to the well-known strongly thrombogenic glass surface and to the endothelic cell heparan sulphate classified as antithrombogenic. The diagram shows clearly that the linseed matrix with and with out PVP addition clearly distinguishes itself in this comparison as the surface which effects the lowest thrombocyte adhesion. Therewith, the linseed oil distinguishes itself as hemocompatible matrix for the coating of implants with contact to blood. Moreover, a further improvement arises if the addition of PVP is omitted because the PVP which is proven as hemocompatible shows in average a slight increase of the thrombocyte adhesion.

1-31. (canceled)

32. Medical product the surface of which comprises at least partially a polymer layer, wherein the polymer layer consists of at least 25% by weight of substances participating in the polymerization reaction and the polymer layer comprises substances, wherein the substances participating in the polymerization reaction contain a linear or branched and a substituted or non substituted alkyl moiety with at least one multiple bond and the substances participating in the polymerization reaction are capable of auto-polymerization.

33. Medical product according to claim 32, wherein the alkyl moiety containing at least one multiple bond has 7 to 50 carbon atoms.

34. Medical product according to claim 32, wherein the substances containing at least one alkyl moiety with at least one multiple bond are covalently linked with each other via polymerization of the at least one multiple bond.

35. Medical product according to claim 32, wherein the substances containing at least one alkyl moiety with at least one multiple bond are chosen from the group comprising fatty acids, fatty acid esters, fatty acid derivatives, ethers, diethers, tetraethers, lipids, oils, fats, glycerides, tri-glycerides, glycol esters, glycerin esters as well as mixtures of the aforementioned substances.

36. Medical product according to claim 35, wherein in the case of the lipids mono- or poly-unsaturated fatty acids and/or mixtures of these unsaturated fatty acids in the form of their triglycerides and/or in non glycerin bound, free form are concerned.

37. Medical product according to claim 36, characterized in that the unsaturated fatty acids are chosen from the group comprising oleic acid, eicosapentaenoic acid, timnodonic acid, docosahexaenoic acid, arachidonic acid, linoleic acid, α-linolenic acid, γ-linolenic acid as well as mixtures of the aforementioned fatty acids.

38. Medical product according to claim 35, characterized in that in the case of the oils linseed oil, hempseed oil, corn oil, walnut oil, rape oil, soy bean oil, sun flower oil, poppy-seed oil, safflower oil, wheat germ oil, grape-seed oil, evening primrose oil, borage oil, black cumin oil, algae oil, fish oil, cod-liver oil and/or mixtures of the aforementioned substances are concerned.

39. Medical product according to claim 38, characterized in that the oils and the mixtures of the oils, respectively, contain an amount of at least 40% by weight of unsaturated fatty acids.

40. Medical product according to claim 32, characterized in that the substances not participating in the polymerization reaction comprise saturated fatty acids, saturated fatty acid esters, saturated fatty acid derivatives, saturated ethers, saturated lipids, lipoids, saturated fats and oils, saturated glycerides, saturated triglycerides, saturated glycol esters, saturated glycerin esters, waxes, biostable or biodegradable polymers or mixtures of the aforementioned substances.

41. Medical product according to claim 40, characterized in that in the case of the saturated fatty acids the long-chain fatty acids beyond a chain length of 12 carbon atoms as well as mixtures thereof and/or natural lipoids such as palm kernel fat, coconut fat as well as their mixtures are concerned.

42. Medical product according to claim 40, characterized in that in the case of the waxes beeswax, carnauba wax, candelilla wax and/or mixtures thereof are concerned.

43. Medical product according to claim 40, characterized in that the biostable polymers are chosen from the group comprising polyacrylic acid and polyacrylates such as polymethylmethacrylate, polybutylmethacrylate, polyacrylamide, polyacrylonitriles, polyamides, polyetheramides, polyethylenamine, polyimides, polycarbonates, polycarbourethanes, polyvinylketones, polyvinylhalogenides, polyvinylidenhalogenides, polyvinylethers, polyvinylaromates, polyvinylesters, polyvinylpyrollidones, polyoxymethylenes, polyethylene, polypropylene, polytetrafluoroethylene, polyurethanes, polyolefine elastomeres, polyisobutylenes, EPDM gums, fluorosilicones, carboxymethylchitosanes, polyethyleneterephthalate, polyvalerates, carboxymethylcellulose, cellulose, rayon, rayontriacetates, cellulosenitrates, celluloseacetates, hydroxyethylcellulose, cellulosebutyrates, celluloseacetatebutyrates, ethylvinylacetate copolymers, polysulphones, epoxy resins, ABS resins, EPDM gums, silicones such as polysiloxanes, polyvinylhalogenes and copolymers, celluloseethers, cellulosetriacetates, chitosanes and copolymers and/or mixtures of these substances.

44. Medical product according to claim 40, characterized in that the biodegradable polymers are chosen from the group comprising polyvalerolactones, poly-ε-decalactones, polylactides, polyglycolides, copolymers of the polylactides and polyglycolides, poly-ε-caprolactone, polyhydroxybutanoic acid, polyhydroxybutyrates, polyhydroxyvalerates, polyhydroxybutyrate-co-valerates, poly(1,4-dioxane-2,3-diones), poly(1,3-dioxane-2-one), poly-para-dioxanones, polyanhydrides such as polymaleic anhydrides, polyhydroxymethacrylates, fibrin, polycyanoacrylates, polycaprolactonedimethylacrylates, poly-b-maleic acid, polycaprolactonebutyl-acrylates, multiblock polymers such as for example from oligocaprolactonedioles and oligodioxanonedioles, polyetherester multiblock polymers such as for example PEG and poly(butyleneterephtalates), polypivotolactones, polyglycolic acid trimethyl-carbonates, polycaprolactone-glycolides, poly(g-ethylglutamate), poly(DTH-iminocarbonate), poly(DTE-co-DT-carbonate), poly(bisphenol-A-iminocarbonate), polyorthoesters, polyglycolic acid trimethyl-carbonates, polytrimethylcarbonates, polyiminocarbonates, poly(N-vinyl)-pyrrolidone, polyvinylalcoholes, polyesteramides, glycolated polyesters, polyphosphoesters, polyphosphazenes, poly[p-carboxyphenoxy)propane], polyhydroxypentanoic acid, polyanhydrides, polyethyleneoxide-propyleneoxide, soft polyurethanes, polyurethanes with amino acid moieties in the backbone, polyetheresters such as polyethyleneoxide, polyalkeneoxalates, polyorthoesters as well as their copolymers, carrageenans, fibrinogen, starch, collagen, protein based polymers, polyamino acids, synthetic polyamino acids, zein, modified zein, polyhydroxyalkanoates, pectic acid, actinic acid, modified and non modified fibrin and casein, carboxymethylsulphate, albumin, moreover hyaluronic acid, heparansulphate, heparin, chondroitinesulphate, dextran, b-cyclodextrines and copolymers with PEG and polypropyleneglycol, gummi arabicum, guar, gelatin, collagen, collagen-N-Hydroxysuccinimide, modifications and copolymers and/or mixtures of the aforementioned substances.

45. Medical product according to claim 32, characterized in that the substances not participating in the polymerization reaction comprise antiproliferative, antiinflammatoric and/or antithrombotic active agents chosen from the group comprising sirolimus (rapamycin), everolimus, pimecrolimus, somatostatin, tacrolimus, roxithromycin, dunaimycin, ascomycin, bafilomycin, erythromycin, midecamycin, josamycin, concanamycin, clarithromycin, troleandomycin, folimycin, cerivastatin, simvastatin, lovastatin, fluvastatin, rosuvastatin, atorvastatin, pravastatin, pitavastatin, vinblastine, vincristine, vindesine, vinorelbine, etoposide, teniposide, nimustine, carmustine, lomustine, cyclophosphamide, 4-hydroxycyclophosphamide, estramustine, melphalan, ifosfamide, trofosfamide, chlorambucil, bendamustine, dacarbazine, busulfan, procarbazine, treosulfan, temozolomide, thiotepa, daunorubicin, doxorubicin, aclarubicin, epirubicin, mitoxantrone, idarubicin, bleomycin, mitomycin, dactinomycin, methotrexate, fludarabine, fludarabine-5′-dihydrogenphosphate, cladribine, mercaptopurine, thioguanine, cytarabine, fluorouracil, gemcitabine, capecitabine, docetaxel, carboplatin, cisplatin, oxaliplatin, amsacrine, irinotecan, topotecan, hydroxycarbamide, miltefosine, pentostatin, aldesleukin, tretinoin, asparaginase, pegaspargase, anastrozole, exemestane, letrozole, formestane, aminoglutethimide, adriamycin, azithromycin, spiramycin, cepharantin, smc proliferation inhibitor-2w, epothilone A and B, mitoxantrone, azathioprine, mycophenolatmofetil, c-myc-antisense, b-myc-antisense, betulinic acid, camptothecin, PI-88 (sulfated oligosaccharide), melanocyte stimulating hormone (α-MSH), activated protein C, IL-1β inhibitor, thymosine α-1, fumaric acid and its esters, calcipotriol, tacalcitol, lapachol, β-lapachone, podophyllotoxin, betulin, podophyllic acid 2-ethylhydrazide, molgramostim (rhuGM-CSF), peginterferon α-2b, lenograstim (r-HuG-CSF), filgrastim, macrogol, dacarbazine, basiliximab, daclizumab, selectin (cytokine antagonist), CETP inhibitor, cadherines, cytokinin inhibitors, COX-2 inhibitor, NFkB, angiopeptin, ciprofloxacin, camptothecin, fluoroblastin, monoclonal antibodies, which inhibit the muscle cell proliferation, bFGF antagonists, probucol, prostaglandins, 1,11-dimethoxycanthin-6-one, 1-hydroxy-11-methoxycanthin-6-one, scopoletin, colchicine, NO donors such as pentaerythritol tetranitrate and syndnoeimines, S-nitrosoderivatives, tamoxifen, staurosporine, β-estradiol, α-estradiol, estriol, estrone, ethinylestradiol, fosfestrol, medroxyprogesterone, estradiol cypionates, estradiol benzoates, tranilast, kamebakaurin and other terpenoids, which are applied in the therapy of cancer, verapamil, tyrosine kinase inhibitors (tyrphostines), cyclosporine A, paclitaxel and derivatives thereof such as 6-α-hydroxy-paclitaxel, baccatin, taxotere, synthetically produced as well as from native sources obtained macrocyclic oligomers of carbon suboxide (MCS) and derivatives thereof, mofebutazone, acemetacin, diclofenac, lonazolac, dapsone, o-carbamoylphenoxyacetic acid, lidocaine, ketoprofen, mefenamic acid, piroxicam, meloxicam, chloroquine phosphate, penicillamine, tumstatin, avastin, D-24851, SC-58125, hydroxychloroquine, auranofin, sodium aurothiomalate, oxaceprol, celecoxib, β-sitosterin, ademetionine, myrtecaine, polidocanol, nonivamide, levomenthol, benzocaine, aescin, ellipticine, D-24851 (Calbiochem), colcemid, cytochalasin A-E, indanocine, nocodazole, S 100 protein, bacitracin, vitronectin receptor antagonists, azelastine, guanidyl cyclase stimulator, tissue inhibitor of metal proteinase-1 and -2, free nucleic acids, nucleic acids incorporated into virus transmitters, DNA and RNA fragments, plasminogen activator inhibitor-1, plasminogen activator inhibitor-2, antisense oligonucleotides, VEGF inhibitors, IGF-1; active agents from the group of the antibiotics such as cefadroxil, cefazolin, cefaclor, cefotaxim, tobramycin, gentamycin, penicillins such as dicloxacillin, oxacillin, sulfonamides, metronidazol, antithrombotics such as argatroban, aspirin, abciximab, synthetic antithrombin, bivalirudin, coumadin, enoxaparin, desulphated and N-reacetylated heparin, tissue plasminogen activator, GpIIb/IIIa platelet membrane receptor, factor Xₐ inhibitor antibodies, heparin, hirudin, r-hirudin, PPACK, protamin, sodium salt of 2-methylthiazolidine-2,4-dicarboxylic acid, prourokinase, streptokinase, warfarin, urokinase, vasodilators such as dipyramidole, trapidil, nitroprussides, PDGF antagonists such as triazolopyrimidine and seramin, ACE inhibitors such as captopril, cilazapril, lisinopril, enalapril, losartan, thio-protease inhibitors, prostacyclin, vapiprost, α, β and γ interferon, histamine antagonists, serotonin blockers, apoptosis inhibitors, apoptosis regulators such as p65, NF-kB or Bcl-xL antisense oligonucleotides, halofuginone, nifedipine, tocopherol, vitamin B1, B2, B6 and B12, folic acid, tranilast, molsidomine, tea polyphenols, epicatechin gallate, epigallocatechin gallate, Boswellinic acids and derivatives thereof, leflunomide, anakinra, etanercept, sulfasalazine, etoposide, dicloxacillin, tetracycline, triamcinolone, mutamycin, procainamid, D24851, SC-58125, retinoic acid, quinidine, disopyramide, flecainide, propafenone, sotalol, amidorone, natural and synthetically produced steroids such as bryophyllin A, inotodiol, maquiroside A, ghalakinoside, mansonine, strebloside, hydrocortisone, betamethasone, dexamethasone, non-steroidal substances (NSAIDS) such as fenoprofen, ibuprofen, indomethacin, naproxen, phenylbutazone and other antiviral agents such as acyclovir, ganciclovir and zidovudine, antimycotics such as clotrimazole, flucytosine, griseofulvin, ketoconazole, miconazole, nystatin, terbinafine, antiprozoal agents such as chloroquine, mefloquine, quinine, moreover natural terpenoids such as hippocaesculin, barringtogenol-C21-angelate, 14-dehydroagrostistachin, agroskerin, agrostistachin, 17-hydroxyagrostistachin, ovatodiolids, 4,7-oxycycloanisomelic acid, baccharinoids B1, B2, B3 and B7, tubeimoside, bruceanol A, B and C, bruceantinoside C, yadanziosides N and P, isodeoxyelephantopin, tomenphantopin A and B, coronarin A, B, C and D, ursolic acid, hyptatic acid A, zeorin, iso-iridogermanal, maytenfoliol, effusantin A, excisanin A and B, longikaurin B, sculponeatin C, kamebaunin, leukamenin A and B, 13,18-dehydro-6-α-senecioyloxychaparrin, taxamairin A and B, regenilol, triptolide, moreover cymarin, apocymarin, aristolochic acid, anopterin, hydroxyanopterin, anemonin, protoanemonin, berberine, cheliburin chloride, cictoxin, sinococuline, bombrestatin A and B, cudraisoflavone A, curcumin, dihydronitidine, nitidine chloride, 12-β-hydroxypregnadiene-3,20-dione, bilobol, ginkgol, ginkgolic acid, helenalin, indicine, indicine-N-oxide, lasiocarpine, inotodiol, glycoside 1a, podophyllotoxin, justicidin A and B, larreatin, malloterin, mallotochromanol, isobutyrylmallotochromanol, maquiroside A, marchantin A, maytansine, lycoridicin, margetine, pancratistatin, liriodenine, oxoushinsunine, aristolactam-AII, bisparthenolidine, periplocoside A, ghalakinoside, ursolic acid, deoxypsorospermin, psychorubin, ricin A, sanguinarine, manwu wheat acid, methylsorbifolin, sphatheliachromen, stizophyllin, mansonine, strebloside, akagerine, dihydrousambarensine, hydroxyusambarine, strychnopentamine, strychnophylline, usambarine, usambarensine, berberine, liriodenine, oxoushinsunine, daphnoretin, lariciresinol, methoxylariciresinol, syringaresinol, umbelliferon, afromoson, acetylvismione B, desacetylvismione A, vismione A and B and sulfur containing amino acids such as cystine as well as salts and/or mixtures of the aforementioned active agents.

46. Medical product according to claim 45, characterized in that the active agent is chosen from the group comprising tacrolimus, pimecrolimus, PI-88, paclitaxel and its derivatives, trapidil, α- and β-estradiol, sodium salt of 2-methylthiazolidine-2,4-dicarboxylic acid, macrocyclic carbon suboxide (MCS) and its derivatives, sirolimus, fumaric acid and its esters, activated protein C, interleukin-1β inhibitors and melanocyte-stimulating hormone (α-MSH), cystine, ellipticine, bohemine, indanocine, colcemid and derivatives thereof, methionine as well as salts and/or mixtures of the aforementioned active agents.

47. Medical product according to claim 45, wherein at least one antiproliferative, antiinflammatoric and/or antithrombotic active agent is bound covalently and/or adhesively under and/or in and/or on the polymer layer.

48. Medical product according to claims 45, characterized in that the antiproliferative, antiinflammatoric and/or antithrombotic active agent according to claim 14 is contained in a pharmaceutically active concentration of 0.001 to 10 mg per cm² surface of the medical product.

49. Medical product according to claims 32, wherein the substances for the polymer layer contain a polymerization catalyst in a biocompatible concentration.

50. Method for the biocompatible coating of medical products comprising the steps:
a) providing a surface of a medical product, and
b) deposition of the substances for the polymer layer, and
c) polymerization of the substances containing at least one alkyl moiety with at least one multiple bond by means of exposure to heat, light and/or aerial oxygen and/or by means of one a catalyst contained in a biocompatible concentration.

51. Method for the biocompatible coating of medical products comprising the steps:
a) providing a surface of a medical product, and
a′) deposition of layer of an antiproliferative, antiinflammatoric and/or antithrombotic active agent, and
b) deposition of the substances for the polymer layer, and
c) polymerization of the substances containing at least one alkyl moiety with at least one multiple bond by means of exposure to heat, light and/or aerial oxygen and/or by means of one a catalyst contained in a biocompatible concentration.

52. Method according to claim 50 further comprising the step d):
d) deposition and/or incorporation of a layer of an antiproliferative, antiinflammatoric and/or antithrombotic active agent on the polymer layer.

53. Method according to claim 50 further comprising the step e):
e) deposition of at least another polymerized layer of the polymers selected from the group comprising polyacrylic acid and polyacrylates such as polymethylmethacrylate, polybutylmethacrylate, polyacrylamide, polyacrylonitriles, polyamides, polyetheramides, polyethylenamine, polyimides, polycarbonates, polycarbourethanes, polyvinylketones, polyvinylhalogenides, polyvinylidenhalogenides, polyvinylethers, polyvinylaromates, polyvinylesters, polyvinylpyrollidones, polyoxymethylenes, polyethylene, polypropylene, polytetrafluoroethylene, polyurethanes, polyolefine elastomeres, polyisobutylenes, EPDM gums, fluorosilicones, carboxymethylchitosanes, polyethyleneterephthalate, polyvalerates, carboxymethylcellulose, cellulose, rayon, rayontriacetates, cellulosenitrates, celluloseacetates, hydroxyethylcellulose, cellulosebutyrates, celluloseacetatebutyrates, ethylvinylacetate copolymers, polysulphones, epoxy resins, ABS resins, EPDM gums, silicones such as polysiloxanes, polyvinylhalogenes and copolymers, celluloseethers, cellulosetriacetates, chitosanes, polyvalerolactones, poly-E-decalactones, polylactides, polyglycolides, copolymers of the polylactides and polyglycolides, poly-ε-caprolactone, polyhydroxybutanoic acid, polyhydroxybutyrates, polyhydroxyvalerates, polyhydroxybutyrate-co-valerates, poly(1,4-dioxane-2,3-diones), poly(1,3-dioxane-2-one), poly-para-dioxanones, polyanhydrides such as polymaleic anhydrides, polyhydroxymethacrylates, fibrin, polycyanoacrylates, polycaprolactonedimethylacrylates, poly-b-maleic acid, polycaprolactonebutyl-acrylates, multiblock polymers such as for example from oligocaprolactonedioles and oligodioxanonedioles, polyetherester multiblock polymers such as for example PEG and poly(butyleneterephtalates), polypivotolactones, polyglycolic acid trimethyl-carbonates, polycaprolactone-glycolides, poly(g-ethylglutamate), poly(DTH-iminocarbonate), poly(DTE-co-DT-carbonate), poly(bisphenol-A-iminocarbonate), polyorthoesters, polyglycolic acid trimethyl-carbonates, polytrimethylcarbonates, polyiminocarbonates, poly(N-vinyl)-pyrrolidone, polyvinylalcoholes, polyesteramides, glycolated polyesters, polyphosphoesters, polyphosphazenes, poly[p-carboxyphenoxy)propane], polyhydroxypentanoic acid, polyanhydrides, polyethyleneoxide-propyleneoxide, soft polyurethanes, polyurethanes with amino acid moieties in the backbone, polyetheresters such as polyethyleneoxide, polyalkeneoxalates, polyorthoesters as well as their copolymers, carrageenans, fibrinogen, starch, collagen, protein based polymers, polyamino acids, synthetic polyamino acids, zein, modified zein, polyhydroxyalkanoates, pectic acid, actinic acid, modified and non modified fibrin and casein, carboxymethylsulphate, albumin, moreover hyaluronic acid, heparansulphate, heparin, chondroitinesulphate, dextran, b-cyclodextrines and copolymers with PEG and polypropyleneglycol, gummi arabicum, guar, gelatin, collagen, collagen-N-Hydroxysuccinimide, modifications and copolymers and mixtures of the aforementioned substances on the subjacent layer or of another polymer layer according to the steps b) and c).

54. Method according to claim 50, characterized in that the antiproliferative, antiinflammatoric and/or antithrombotic active agent is bound covalently and/or adhesively in and/or to the respective layer.

55. Method according to claim 50, characterized in that the antiproliferative, antiinfilammatoric and/or antithrombotic active agent is present in a pharmaceutically active concentration of 0.001 to 10 mg per cm² surface of the medical product.

56. Medical product obtainable in accordance with the method according to claim 50.

57. Medical product obtainable in accordance with the method according to claim 51.

58. Medical product according to one of the claim 1, characterized in that in the case of the medical product a stent is concerned.

59. Medical product according to claim 58, wherein the stent is suitable to prevent or to reduce restenosis.

60. Medical product according to claim 58, wherein the stent is suitable to continuously release at least one antiproliferative, antiinflammatoric, antiangiogenic and/or antithrombotic active agent.

61. Method according to claim 51 further comprising the step d):
d) deposition and/or incorporation of a layer of an antiproliferative, antiinflammatoric and/or antithrombotic active agent on the polymer layer.

62. Method according to claim 51 further comprising the step C):
e) deposition of at least another polymerized layer of the polymers selected from the group comprising polyacrylic acid and polyacrylates such as polymethylmethacrylate, polybutylmethacrylate, polyacrylamide, polyacrylonitriles, polyamides, polyetheramides, polyethylenamine, polyimides, polycarbonates, polycarbourethanes, polyvinylketones, polyvinylhalogenides, polyvinylidenhalogenides, polyvinylethers, polyvinylaromates, polyvinylesters, polyvinylpyrollidones, polyoxymethylenes, polyethylene, polypropylene, polytetrafluoroethylene, polyurethanes, polyolefine elastomeres, polyisobutylenes, EPDM gums, fluorosilicones, carboxymethylchitosanes, polyethyleneterephthalate, polyvalerates, carboxymethylcellulose, cellulose, rayon, rayontriacetates, cellulosenitrates, celluloseacetates, hydroxyethylcellulose, cellulosebutyrates, celluloseacetatebutyrates, ethylvinylacetate copolymers, polysulphones, epoxy resins, ABS resins, EPDM gums, silicones such as polysiloxanes, polyvinylhalogenes and copolymers, celluloseethers, cellulosetriacetates, chitosanes, polyvalerolactones, poly-ε-decalactones, polylactides, polyglycolides, copolymers of the polylactides and polyglycolides, poly-ε-caprolactone, polyhydroxybutanoic acid, polyhydroxybutyrates, polyhydroxyvalerates, polyhydroxybutyrate-co-valerates, poly(1,4-dioxane-2,3-diones), poly(1,3-dioxane-2-one), poly-para-dioxanones, polyanhydrides such as polymaleic anhydrides, polyhydroxymethacrylates, fibrin, polycyanoacrylates, polycaprolactonedimethylacrylates, poly-b-maleic acid, polycaprolactonebutyl-acrylates, multiblock polymers such as for example from oligocaprolactonedioles and oligodioxanonedioles, polyetherester multiblock polymers such as for example PEG and poly(butyleneterephtalates), polypivotolactones, polyglycolic acid trimethyl-carbonates, polycaprolactone-glycolides, poly(g-ethylglutamate), poly(DTH-iminocarbonate), poly(DTE-co-DT-carbonate), poly(bisphenol-A-iminocarbonate), polyorthoesters, polyglycolic acid trimethyl-carbonates, polytrimethylcarbonates, polyiminocarbonates, poly(N-vinyl)-pyrrolidone, polyvinylalcoholes, polyesteramides, glycolated polyesters, polyphosphoesters, polyphosphazenes, poly[p-carboxyphenoxy)propane], polyhydroxypentanoic acid, polyanhydrides, polyethyleneoxide-propyleneoxide, soft polyurethanes, polyurethanes with amino acid moieties in the backbone, polyetheresters such as polyethyleneoxide, polyalkeneoxalates, polyorthoesters as well as their copolymers, carrageenans, fibrinogen, starch, collagen, protein based polymers, polyamino acids, synthetic polyamino acids, zein, modified zein, polyhydroxyalkanoates, pectic acid, actinic acid, modified and non modified fibrin and casein, carboxymethylsulphate, albumin, moreover hyaluronic acid, heparansulphate, heparin, chondroitinesulphate, dextran, b-cyclodextrines and copolymers with PEG and polypropyleneglycol, gummi arabicum, guar, gelatin, collagen, collagen-N-Hydroxysuccinimide, modifications and copolymers and mixtures of the aforementioned substances on the subjacent layer or of another polymer layer according to the steps b) and c).

63. Method according to claim 51, characterized in that the antiproliferative, antiinflammatoric and/or antithrombotic active agent is bound covalently and/or adhesively in and/or to the respective layer.

64. Method according to claim 51, characterized in that the antiproliferative, antiinflammatoric and/or antithrombotic active agent is present in a pharmaceutically active concentration of 0.001 to 10 mg per cm² surface of the medical product.

65. Medical product according to one of the claim 56, characterized in that in the case of the medical product a stent is concerned.

66. Medical product according to claim 65, wherein the stent is suitable to prevent or to reduce restenosis.

67. Medical product according to claim 65, wherein the stent is suitable to continuously release at least one antiproliferative, antiinflammatoric, antiangiogenic and/or antithrombotic active agent.

68. Medical product according to one of the claim 57, characterized in that in the case of the medical product a stent is concerned.

69. Medical product according to claim 68, wherein the stent is suitable to prevent or to reduce restenosis.

70. Medical product according to claim 68, wherein the stent is suitable to continuously release at least one antiproliferative, antiinflammatoric, antiangiogenic and/or antithrombotic active agent.
